(19) United States
(12) Patent Application Publication
(10) Pub. No.: US 2023/0248778 A1
(43) Pub. Date: Aug. 10, 2023

(54) THERAPEUTIC USE OF OF CELL-FREE FAT EXTRACT FOR PULMONARY DISEASES

(71) Applicant: SHANGHAI SEME CELL TECHNOLOGY CO., LTD., Shanghai (CN)

(72) Inventors: Wenjie ZHANG, Shanghai (CN); Ziyou YU, Shanghai (CN); Xifan HOU, Shanghai (CN)

(21) Appl. No.: 18/001,259

(22) PCT Filed: Jun. 11, 2021
(86) PCT No.: PCT/CN2021/099865

§ 371 Date: Dec. 8, 2022

(30) Foreign Application Priority Data
Jun. 12, 2020 (CN) 202010537360.0

(51) Int. Cl. A61K 35/35; C12N 5/077; A61P 11/00

(52) U.S. Cl. CPC A61K 35/35; A61P 11/00; C12N 5/0653

(57) ABSTRACT

Disclosed are a cell-free fat extract and a preparation method therefor and the method of use thereof. The cell-free fat extract is used for preparing a composition or a preparation, wherein the composition or preparation is used in a method of: preventing and/or treating acute respiratory distress syndrome and/or acute lung injury, preventing and/or treating hypoxemia, improving pulmonary tissue inflammations, improving pulmonary tissue injuries, preventing and/or treating systemic inflammatory response syndrome, and preventing and/or treating multiple organ failure.

### TECHNICAL FIELD

The invention relates to the field of medicine, in particular to the therapeutic use of cell-free fat extract for pulmonary diseases.

### BACKGROUND

Acute lung injury (ALI) is a disease caused by various factors such as damage to alveolar epithelial cells, interstitial capillary damage, and disruption of the alveolar and capillary barriers, resulting in edema and inflammatory cell infiltration in the alveoli and pulmonary interstitium, with dyspnea and hypoxemia as the main clinical manifestations. It is estimated that it accounts for about 10% of intensive care unit admissions worldwide, with a mortality rate of more than 40%. Acute respiratory distress syndrome (ARDS) is an acute hypoxic respiratory failure caused by acute diffuse damage to the lung parenchyma in the most severe stage of acute lung injury, characterized by clinical manifestations of progressive dyspnea and intractable hypoxemia. Currently, the clinical treatment of ALI/ARDS is mainly through mechanical ventilation for respiratory support and glucocorticoids and pulmonary vasodilators due to its high morbidity and mortality rate and the lack of treatment methods, however, current treatments are not effective in reducing mortality in ALI/ARDS, and there is a lack of effective therapeutic drugs. Therefore, there is a need in the art to develop a drug that can effectively treat ALI/ARDS.

### SUMMARY OF THE INVENTION

The purpose of the present invention is to provide a use of cell-free fat extract in the treatment of pulmonary diseases such as ALI/ARDS.

The first aspect of the present invention provides a use of cell-free fat extract in the manufacture of a composition or preparation for one or more uses selected from the group consisting of: (i) prevention and/or treatment of acute respiratory distress syndrome and/or acute lung injury;(ii) prevention and/or treatment of hypoxemia;(iii) improvement of pulmonary tissue inflammation;(iv) improvement of lung tissue damage;(v) prevention and/or treatment of systemic inflammatory response syndrome;(vi) prevention and/or treatment of multiple organ failure.

In another preferred embodiment, the prevention and/or treatment of acute respiratory distress syndrome and/or acute lung injury comprises prevention and/or treatment in one or more ways selected from the group consisting of:
(i) increasing blood oxygen level;
(ii) improving lung tissue inflammation;
(iii) improving pulmonary tissue injury; and/or
(iv) improving respiratory function.

In another preferred embodiment, the prevention and/or treatment of hypoxemia comprises an increase in blood oxygen level.

In another preferred embodiment, the increasing blood oxygen level comprises increasing blood oxygen partial pressure and/or increasing blood oxygen saturation.

In another preferred embodiment, the improvement of lung tissue inflammation comprises reducing inflammatory cell infiltration in the lung.

In another preferred embodiment, the inflammatory cells are selected from the group consisting of white blood cells, neutrophils, lymphocytes, monocytes, and combinations thereof.

In another preferred embodiment, the improvement of pulmonary tissue injury includes one or more ways selected from the group consisting of:
(iv-1) reduction of intrapulmonary inflammatory cell infiltration;
(iv-2) inhibition of intra-alveolar macrophage aggregation;
(iv-3) inhibition of alveolar wall thickening;
(iv-4) inhibition of alveolar and/or perialveolar hemorrhage; and/or
(iv-5) inhibition of intra-alveolar fibrin-like material deposition.

In another preferred embodiment, the hypoxemic patient suffers acute respiratory distress syndrome and/or acute lung injury.

In another preferred embodiment, the patient with lung tissue inflammation suffers acute respiratory distress syndrome and/or acute lung injury.

In another preferred embodiment, the patient with pulmonary tissue injury suffers acute respiratory distress syndrome and/or acute lung injury.

In another preferred embodiment, the cell-free fat extract is a cell-free fat extract obtained from fat in human or non-human mammals.

In another preferred embodiment, the non-human mammal is a monkey, an orangutan, a cow, a pig, a dog, a sheep, a rat or a rabbit.

In another preferred embodiment, the composition or preparation comprises a pharmaceutical composition or preparation, a food composition or preparation, a health care composition or preparation, or a dietary supplement.

In another preferred embodiment, the composition or preparation further comprises a pharmaceutically, food, health care product or a dietary acceptable carrier.

In another preferred embodiment, the dosage form of the composition or preparation is an oral preparation, a topical preparation or an injection preparation.

In another preferred embodiment, the injection preparation is an intravenous injection preparation.

In another preferred embodiment, the composition or preparation is administered by topical, local, or subcutaneous injection.

In another preferred embodiment, the cell-free fat extract is free of cells and free of lipid droplets. In another preferred embodiment, the lipid droplets are oil droplets released after the fat cells are broken.

In another preferred embodiment, the “free of lipid droplets” means that the volume of oil droplets in the cell-free fat extract is less than 1%, preferably less than 0.5%, more preferably less than 0.1% in total liquid percentage.

In another preferred embodiment, the cells are selected from the group consisting of endothelial cells, adipose stem cells, macrophagocytic cells, stromal cells.

In another preferred embodiment, the “cell-free” means that the average number of cells in 1ml of cell-free fat extract is ≤ 1, preferably ≤ 0.5, more preferably ≤ 0.1, or 0.

In another preferred embodiment, the cell-free fat extract is a naturally obtained nano-fat extract with additive-free.

In another preferred embodiment, the “additive-free” means that no solution, solvent, small molecule, chemical agent, and biological additive are added during the preparation of the fat extract except for the rinsing step.

In another preferred embodiment, the fat extract is obtained by centrifuging the fat tissue after emulsification.

In another preferred embodiment, the fat extract contains, but is not limited to, one or more components selected from the group consisting of growth factors IGF-1, BDNF, GDNF, TGF-β, HGF, bFGF, VEGF, PDGF, EGF, NT-3, GH, G-CSF, and combinations thereof.

In another preferred embodiment, the cell-free fat extract contains one or more components selected from the group consisting of IGF-1, BDNF, GDNF, TGF-β, HGF, bFGF, VEGF, TGF-β1, HGF, PDGF, EGF, NT-3, GH, G-CSF, and combinations thereof.

In another preferred embodiment, the cell-free fat extract contains, but is not limited to, one or more components selected from the group consisting of IGF-1, BDNF, GDNF, bFGF, VEGF, TGF-β1, HGF, PDGF, and combinations thereof.

In another preferred embodiment, in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of the IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20: 1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3: 1, preferably 0.2-2:1, more preferably 0.4-1.5:1, and most preferably 0.7-1.2:1.

In another preferred embodiment, the cell-free fat extract is a liquid.

In another preferred embodiment, the cell-free fat extract is prepared by the following method:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano fat);
(5) centrifuging the emulsified fat mixture, thereby obtaining an intermediate liquid layer, i.e. a fat primary extract; and
(6) filtering and degerming the fat primary extract, thereby obtaining a cell-free fat extract.

The second aspect of the present invention provides a method for preparing cell-free fat extract, and the method comprises the steps of:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano fat);
(5) centrifuging the emulsified fat mixture, thereby obtaining an intermediate liquid layer, i.e. a fat primary extract; and
(6) filtering and degerming the fat primary extract, thereby obtaining a cell-free fat extract.

In another preferred embodiment, in step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, the temperature of the centrifugation is 2-6° C.

In another preferred embodiment, in the step (4), the emulsification is mechanical emulsification.

In another preferred embodiment, the mechanical emulsification is performed by repeated blowing by a syringe (e. g., blowing 20-200 times, preferably 20-150 times, more preferably 20-100 times, more preferably 30-50 times).

In another preferred embodiment, the blowing method is that two 10 ml injection syringes are connected to a tee tube and repeatedly push at a constant speed.

In another preferred embodiment, in the step (4), the emulsification is a method of crushing by a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from -50° C. to -120° C., preferably from -60° C. to -100° C., more preferably from -70° C. to -90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i. e., an intermediate liquid layer), and the fourth layer is a cell/tissue debris precipitation layer.

In another preferred embodiment, in step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the fat extract.

In another preferred embodiment, in the step (6), the filtering and degerming are carried out through a filter (such as a 0.22 µm microporous filter membrane).

In another preferred embodiment, the filter is a microporous membrane filter.

In another preferred embodiment, the pore size of the microporous filter membrane is 0.05-0.8 µm, preferably 0.1-0.5 µm, more preferably 0.1-0.4 µm, more preferably 0.15-0.3 µm, more preferably 0.2-0.25 µm, and most preferably 0.22 µm.

In another preferred embodiment, in the step (6), the filtering and degerming is carried out by first filtering through a first filter that can filter cells, and then through a second filter(such as a 0.22 µm filter) that can filter pathogens (such as bacteria).

In another preferred embodiment, the step (6) further includes subpackaging the fat extract to form a subpackaging product. The subpacked extract can be stored at -20° C. for later use; it can be used directly after thawing at low temperature (e. g. -4° C.) or at normal temperature, or stored at low temperature (e. g. 4° C.) for a period of time after thawing.

The third aspect of the present invention provides a cell-free fat extract, the cell-free fat extract is obtained by the method described in the second aspect of the present invention.

The fourth aspect of the present invention provides a composition or preparation, and the composition or preparation comprises (a) a cell-free fat extract as described in the third aspect of the present invention; and (b) a pharmaceutically, food, health care product or dietary acceptable carrier or excipient.

In another preferred embodiment, the dosage form of the composition or preparation is a powder, a granule, a capsule, an injection, a tincture, an oral liquid, a tablet or a lozenge.

In another preferred embodiment, the injectable agent is an intravenous or intramuscular injection.

In another preferred embodiment, the dosage form of the composition or preparation is a solid dosage form, a semi-solid dosage form, or a liquid dosage form, such as a solution, gel, cream, emulsion, ointments, cream, paste, cake, powder, patch, etc.

In another preferred embodiment, the percentage by mass of the cell-free fat extract in the composition or preparation is 5 wt%, preferably 1-20 wt%, based on the total weight of the cosmetic composition.

The fifth aspect of the present invention provides a method for preparing a composition or preparation according to the fourth aspect of the present invention, and the method comprises the step of mixing the cell-free fat extract according to the third aspect of the present invention with a pharmaceutically, food, health care product or dietary acceptable carrier or excipient to form the composition or preparation.

The sixth aspect of the present invention provides a method for (i) preventing and/or treating acute respiratory distress syndrome and/or acute lung injury; (ii) preventing and/or treating hypoxemia; (iii) improving pulmonary tissue inflammation; (iv) improving pulmonary tissue injury; (v) preventing and/or treating systemic inflammatory response syndrome; (vi) preventing and/or treating multiple organ failure, administering the cell-free fat extract described in the third aspect of the present invention to a subject in need thereof.

In another preferred embodiment, the subject is a human or non-human mammal.

In another preferred embodiment, the non-human mammal comprises a rodent, such as a rat, a mouse.

It should be understood that within the scope of the present invention, the above-mentioned technical features of the present invention and the technical features specifically described in the following (such as embodiments) can be combined with each other to form a new or preferred technical solution. Limited to space, it is not repeated herein.

### DESCRIPTION OF THE DRAWINGS

FIG. 1 shows the survival rate of rats in ARDS model.

FIG. 2 shows arterial oxygen partial pressure and oxygen saturation of model rats (M±SD,* p<0.05, **p<0.01).

FIG. 3 shows the count and classification of inflammatory cells in alveolar lavage fluid (* p<0.05, **p<0.01).

FIG. 4 shows HE staining for lung histopathology.

### DETAILED DESCRIPTION OF EMBODIMENTS

After extensive and in-depth research, the present inventors have for the first time developed a cell-free fat extract that can effectively prevent and/or treat acute respiratory distress syndrome, acute lung injury, hypoxemia, pulmonary tissue inflammation, pulmonary tissue injury, systemic inflammatory response syndrome and multiple organ failure. On this basis, the present invention is completed.

### Terms

Unless otherwise defined, all technical and scientific terms used herein have the same meaning as generally understood by those skilled in the art to which the present invention belongs.

As used herein, the terms “include”, “contain” and “comprise” are used interchangeably, including not only open definitions, but also semi-closed, and closed definitions. In other words, the terms include “consisting of” and “consisting essentially”.

In the present invention, the terms “acute lung injury” and “ALI” are used interchangeably.

In the present invention, the terms “acute respiratory distress syndrome” and “ARDS” are used interchangeably.

In the present invention, the terms “systemic inflammatory response syndrome” and “SIRS” are used interchangeably.

In the present invention, the terms “multiple organ failure” and “MODS” are used interchangeably.

In the present invention, the term “prevention” means a method of preventing the onset of a disease and/or its attendant symptoms or protecting a subject from developing the disease. The “prevention” used herein also includes delaying the onset of the disease and/or its accompanying symptoms and reducing the risk of disease in the subject.

The “treatment” described in the present invention includes delaying and terminating the progression of the disease, or eliminating the disease, and does not require 100% inhibition, elimination and reversal. In some embodiments, the composition or pharmaceutical composition of the present invention reduces, inhibits and/or reverses diabetes, for example, by at least about 10%, at least about 30%, at least about 50%, or at least about 80%, compared to the level observed in the absence of the cell-free fat extract, composition, kit, food or health care kit, active ingredient combination described herein.

As used herein, “improvement” includes prevention, treatment, mitigation, reversal and mitigation, etc.

As used herein, the term “IGF-1” is called insulin-like growth factors-1.

As used in the text, the term “BDNF” is called brain-derived neurotrophic factor (BDNF).

As used in the text, the term “GDNF” is called glial cell line-derived neurotrophic factor.

As used in the text, the term “bFGF” is called basic fibroblast growth factor.

As used in the text, the term “VEGF” is called vascular endothelial growth factor.

As used in the text, the term “TGF-β1” is called transforming growth factor-β1.

As used in the text, the term “HGF” is called hepatocyte growth factor.

As used in the text, the term “PDGF” is called platelet-derived growth factor.

As used in the text, the term “EGF” is called Epidermal Growth Factor.

As used in the text, the term “NT-3” is called neurotrophins-3.

As used in the text, the term “GH” is called Growth Hormone .

As used in the text, the term “G-CSF” is called granulocyte colony stimulating factor.

### Cell Free Fat Extract (CEFFE) and Preparation Method Thereof

As used herein, the terms “cell-free fat extract of the present invention”, “extract of the present invention”, “fat extract of the present invention” and the like are used interchangeably to refer to an extract (or extract liquid) derived from fatty tissue prepared without adding any solutions, solvents, small molecules, chemicals, and biological additives during the preparation of the fat extract (other than the rinsing step). A typical process for preparing an extract of the present invention is as described above in the second aspect of the present invention. In addition, it should be understood that although the extract of the present invention does not need to add any additives (or additive ingredients) during the preparation process, some or a small amount of a safe substance (such as a small amount of water) that does not negatively or adversely affect the activity of the extract of the present invention can also be added.

The cell-free fat extract of the present invention can be derived from human fatty tissue, which is purified from nano-fat by removing oil and cell/extracellular matrix parts after centrifugation, and is a cell-free, easy-to-prepare liquid, and rich in various growth factors.

In a preferred embodiment of the present invention, the cell-free fat extract is a cell-free fat extract liquid.

The cell-free fat extract described in the present invention may include a variety of cytokines. Typically, the cell-free fat extract comprises one or more of IGF-1, BDNF, GDNF, TGF-β, HGF, bFGF, VEGF, TGF-β1, PDGF, EGF, NT-3, GH, and G-CSF.

In another preferred embodiment, the cell-free fat extract contains, but is not limited to, one or more components selected from the group consisting of IGF-1, BDNF, GDNF, bFGF, VEGF, TGF-β1, HGF, PDGF, and combinations thereof.

In another preferred embodiment, in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of the IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20:1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β1 to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3:1, preferably 0.2-2:1, more preferably 0.4-1.5:1, and most preferably 0.7-1.2:1.

Preferably, the cell-free fat extract of the present invention is obtained by the method as described above in the second aspect of the present invention. Typically, the cell-free fat extract described in the present invention is prepared by the following methods:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano fat);
(5) centrifuging the emulsified fat mixture, thereby obtaining an intermediate liquid layer, i.e. a fat primary extract; and
(6) filtering and degerming the fat primary extract, thereby obtaining a cell-free fat extract.

In another preferred embodiment, in step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, in the step (4), the emulsification is mechanical emulsification.

In another preferred embodiment, the mechanical emulsification is performed by repeated blowing by a syringe (e. g., blowing 20-200 times, preferably 20-150 times, more preferably 20-100 times, more preferably 30-50 times).

In another preferred embodiment, the blowing method is that two 10 ml injection syringes are connected to a tee tube and repeatedly push at a constant speed.

In another preferred embodiment, in the step (4), the emulsification is a method of crushing by a tissue homogenizer.

In another preferred embodiment, the step (5) further includes freezing and thawing the emulsified fat mixture before the centrifugation treatment.

In another preferred embodiment, the thawed mixture is used for centrifugation after freezing and thawing treatment.

In another preferred embodiment, the freezing temperature is from -50° C. to -120° C., preferably from -60° C. to -100° C., more preferably from -70° C. to -90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fatty tissue layer, the third layer is a liquid layer (i. e., an intermediate liquid layer), and the fourth layer is a cell/tissue debris precipitation layer.

In another preferred embodiment, in step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filter pack is capable of removing fat cells from the fat extract.

In another preferred embodiment, in the step (6), the filtering and degerming are carried out through a filter (such as a 0.22 µm microporous filter membrane).

In another preferred embodiment, the filter is a microporous membrane filter.

In another preferred embodiment, the pore size of the microporous filter membrane is 0.05-0.8 µm, preferably 0.1-0.5 µm, more preferably 0.1-0.4 µm, more preferably 0.15-0.3 µm, more preferably 0.2-0.25 µm, and most preferably 0.22 µm.

In another preferred embodiment, in the step (6), the filtering and degerming is carried out by first filtering through a first filter that can filter cells, and then through a second filter (such as a 0.22 µm filter) that can filter pathogens (such as bacteria).

In another preferred embodiment, the step (6) further includes subpackaging the fat extract to form a subpackaging product. The subpacked extract can be stored at -20° C. for later use; it can be used directly after thawing at low temperature (e. g. -4° C.) or at normal temperature, or stored at low temperature (e. g. 4° C.) for a period of time after thawing.

### Use

The cell-free fat extract described in the present invention can effectively prevent and/or treat acute respiratory distress syndrome, acute lung injury, hypoxemia, pulmonary tissue inflammation, pulmonary tissue injury, systemic inflammatory response syndrome and/or multiple organ failure.

Typically, the cell-free fat extract of the present invention comprises one or more uses selected from the group consisting of: (i) prevention and/or treatment of acute respiratory distress syndrome and/or acute lung injury;(ii) prevention and/or treatment of hypoxemia;(iii) improvement of pulmonary tissue inflammation;(iv) improvement of pulmonary tissue injury;(v) prevention and/or treatment of systemic inflammatory response syndrome; and/or (vi) prevention and/or treatment of multiple organ failure.

In another preferred embodiment, the prevention and/or treatment of acute respiratory distress syndrome and/or acute lung injury comprises prevention and/or treatment in one or more ways selected from the group consisting of:
(ii) increasing blood oxygen levels;
(ii) improving of lung tissue inflammation;
(iii) improving pulmonary tissue injury; and/or
(iv) improving respiratory function.

In another preferred embodiment, the prevention and/or treatment of hypoxemia comprises an increase in blood oxygen level.

In another preferred embodiment, the increasing blood oxygen level comprises increasing blood oxygen partial pressure and/or increasing blood oxygen saturation.

In another preferred embodiment, the improvement of lung tissue inflammation comprises reducing inflammatory cell infiltration in the lung.

In another preferred embodiment, the inflammatory cells include, but are not limited to, white blood cells, neutrophils, lymphocytes, monocytes, or combinations thereof.

In another preferred embodiment, the improvement of pulmonary tissue injury includes one or more methods selected from the group consisting of:
(iii-1) reduction of intrapulmonary inflammatory cell infiltration;
(iii-2) inhibition of intra-alveolar macrophage aggregation;
(iii-3) inhibition of alveolar wall thickening;
(iii-4) inhibition of alveolar and/or perialveolar hemorrhage; and/or
(iii-5) inhibition of intra-alveolar fibrin-like material deposition.

In another preferred embodiment, the hypoxemic patient has acute respiratory distress syndrome and/or acute lung injury.

In another preferred embodiment, the patient with lung tissue inflammation has acute respiratory distress syndrome and/or acute lung injury.

In another preferred embodiment, the patient with pulmonary tissue injury has acute respiratory distress syndrome and/or acute lung injury.

The present invention also provides a method for (i) preventing and/or treating acute respiratory distress syndrome and/or acute lung injury; (ii) preventing and/or treating hypoxemia; (iii) improving pulmonary tissue inflammation; (iv) improving pulmonary tissue injury; (v) preventing and/or treating systemic inflammatory response syndrome; and/or (vi) preventing and/or treating multiple organ failure, wherein the method comprises the step of: administering the cell-free fat extract described in the present invention to a subject in need thereof.

In another preferred embodiment, the subject is a human or non-human mammal.

In another preferred embodiment, the non-human mammal comprises a rodent, such as a rat, a mouse.

### Composition and Application

The compositions described herein include, but are not limited to, pharmaceutical compositions, food compositions, health-care compositions, dietary supplements, and the like.

Typically, the cell-free fat extract of the present invention can be prepared as pharmaceutical compositions in dosage forms such as tablets, capsules, powders, microgranule, solutions, lozenges, jellies, cream, spiritus, suspensions, tinctures, mud dressings, liniment, lotions, and aerosols, etc. Pharmaceutical compositions can be prepared by commonly known preparation techniques, and suitable pharmaceutical additives can be added to the drug.

The compositions of the present invention can also include pharmaceutically, food, health care product or dietary acceptable carriers. “Pharmaceutically, food, health care product or dietary acceptable carriers” means one or more compatible solid or liquid filler or gel substances that are suitable for human use and must have sufficient purity and sufficiently low toxicity. “Compatibility” herein refers to the ability of components of a composition to blend with the compounds of the invention and with each without significantly reducing the efficacy of the compounds. Examples of pharmaceutically, food, health care product or dietary acceptable carriers include cellulose and its derivatives (such as sodium carboxymethyl cellulose, sodium ethyl cellulose, cellulose acetate, etc.), gelatin, talc, solid lubricants (such as stearic acid, Magnesium stearate), calcium sulfate, vegetable oil (such as soybean oil, sesame oil, peanut oil, olive oil, etc.), polyols (such as propylene glycol, glycerin, mannitol, sorbitol, etc.), emulsifier (such as Tween®), wetting agents (such as sodium dodecyl sulfate), colorants, flavoring agents, stabilizers, antioxidants, preservatives, non-thermal raw water, etc.

The methods of administration of the compositions of the present invention are not particularly limited, and representative methods of administration include, but are not limited to, oral, parenteral (intravenous, intramuscular), topical administration, preferably oral administration and injection administration.

Solid dosage forms for oral administration include capsules, tablets, pills, powders and granules. In these solid dosage forms, the active compounds is mixed with at least one conventional inert excipient (or carrier), such as sodium citrate or dicalcium phosphate, or mixed with:(a) fillers or compatibilizers, e.g., starch, lactose, sucrose, glucose, mannitol and silicic acid;(b) binders, e.g., hydroxymethylcellulose, alginate, gelatin, polyvinylpyrrolidone, sucrose, and gum arabic; (c) humectants, e.g., glycerol;(d) disintegrants, e.g., agar, calcium carbonate, potato starch or tapioca starch, alginic acid, certain complex silicates, and sodium carbonate;(e) dissolution-retarding agents, e.g., paraffin;(f) absorption accelerators, e.g., quaternary amine compounds;(g) wetting agents, e.g., cetearyl alcohol and glycerol monostearate; (h) sorbents, e.g., kaolin; and (i) lubricants, e.g., talc, calcium stearate, magnesium stearate, solid polyethylene glycol, sodium dodecyl sulfate, or mixtures thereof. In capsules, tablets and pills, dosage forms may also contain buffers.

Solid dosage forms such as tablets, sugar pills, capsules and granules may be prepared using coating and shell materials such as casing and other materials well known in the art. They can contain opaque agents.

Liquid dosage forms for oral administration include pharmaceutically acceptable emulsions, solutions, suspensions, syrups or tinctures. In addition to the active compounds, the liquid dosage form may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or mixtures thereof.

In addition to these inert diluents, the composition may also contain auxiliaries such as wetting agents, emulsifiers, suspending agents, sweeteners, flavoring agents and flavors.

In addition to the active ingredient, the suspension may comprise suspending agents, such as ethoxylated isooctadecanol, polyoxyethylene sorbitol and dehydrated sorbitol esters, microcrystalline cellulose, methanolic aluminum, agar, and any mixtures thereof.

The composition for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

Dosage forms of the compounds of the invention for topical administration include ointments, powder, patches, sprays and inhalants. The active ingredient is mixed under sterile conditions with a physiologically acceptable carrier and any preservatives buffers or propellants as may be required.

The cell-free fat extract of the present invention can be administered alone, or in combination with other drugs for the prevention and/or treatment of fatty liver and/or its complications.

When the composition is administered, a safe and effective amount of the cell-free fat extract of the present invention is applied to a human or non-human animal in need of treatment (e. g., rat, mouse, dog, cat, cow, chicken, duck, etc.) at a dose that is pharmaceutically, food or dietary acceptable to the effective administration. As used herein, the term “safe and effective amount” refers to an amount that produces function or activity to humans and/or animals and is acceptable to humans and/or animals. Those ordinary skilled in the art will understand that the “safe and effective amount” described may vary depending on the form of the pharmaceutical composition, the route of administration, the excipient of the drug used, the severity of the disease, and the combination with other drugs. For example, for a person of 60 kg body weight, the daily dose is usually 0.1 to 1000 mg, preferably 1 to 600 mg, more preferably 2 to 300 mg. Of course, the specific dosage should also consider the route of administration, the patient’s health and other factors, which are within the skill range of skilled doctors.

### The Main Advantages of the Present Invention Include

The invention for the first time found that cell-free fat extract can effectively prevent and/or treat acute respiratory distress syndrome, acute lung injury, hypoxemia, pulmonary tissue inflammation, pulmonary tissue injury, systemic inflammatory response syndrome and multiple organ failure.

The present invention is further described below in conjunction with specific examples. It should be understood that these examples are only used to illustrate the present invention and not to limit the scope of the present invention. In the following examples, the test methods without specific conditions are usually in accordance with conventional conditions or the conditions recommended by the manufacturer. Unless otherwise specified, percentages and parts are calculated by weight.

### Example 1

### 1.1. Preparation of Cell Free Fat Extract (CEFFE)

Fat is obtained by volunteers with informed consent. The preparation method of cell free fat tissue extract is as follows:

Fatty tissue was obtained from 6 healthy women who underwent conventional liposuction, with an average age of 31 years (24-36 years). After anesthesia with local injection of swelling solution, a 3 mm liposuction aspiration cannula with a large lateral hole (2 mm × 7 mm) connected to a 20 mL syringe was used, and the obtained fat was manually aspirated radially under negative pressure, and the fat was left upright and stationary, and after removal of the swelling solution, it was rinsed 3 times with saline.

The rinsed fatty tissue was taken, placed in a centrifuge tube, then placed in a centrifuge, and centrifuged at 1200 g 4° C. for 3 minutes to obtain a layered mixture.

For the layered mixture, the upper oil layer and the lower water layer were removed and the intermediate layer (i.e. the fat layer containing fat cells) was collected.

For said intermediate layer, two 10 ml syringes connected to a tee tube were pushed repeatedly and uniformly for 30 times, thus performing mechanical emulsification and obtaining a mechanically emulsified fat mixture (also called nano-fat).

The said mechanically emulsified fat mixture was placed into a -80° C. refrigerator for freezing, and then thawed in a 37° C. water bath, and after a single freeze-thaw cycle, the thawed fat mixture was centrifuged at 1200 g 4° C. for 5 minutes to obtain a layered mixture, which was divided into 4 layers, the first layer being the oil layer, the second layer being the residual fatty tissue layer, the third layer being the liquid layer, and the fourth layer being the cell/tissue debris precipitation layer, the oil layer and the residual fatty tissue layer were removed and the liquid layer was aspirated, avoiding contamination of the cellular/tissue debris precipitation layer during the aspiration process, resulting in a fat primary extract.

The obtained fat primary extract was filtered and degermed through a 0.22 µm filter, thereby sterilizing and removing any live cells that may have been mixed, resulting in a cell-free fat extract that was subpackaged and stored frozen at -20° C. and thawed at 4° C. when used. The content of cytokines, including IGF-1, BDNF, GDNF, bFGF, VEGF, TGF-β1, HGF and PDGF of the obtained cell-free fat extract, were detected by ELISA immunosorbent assay kit. The average concentrations of 6 samples were as follows: IGF-1 (9840.6 pg/ml), BDNF (1764.5 pg/ml), GDNF (1831.9 pg/ml), bFGF (242.3 pg/ml), VEGF (202.9 pg/ml), TGF-β1 (954.5 pg/ml), HGF (898.4 pg/ml), and PDGF (179.9 pg/ml).

### 1.2 Establishment and Grouping of ARDS Model in Rats

Lipopolysaccharide (LPS) was used for the construction of ARDS (acute respiratory distress syndrome) rat model

8-week-old male SD rats were purchased from Beijing Weitong Lihua Experimental Animal Technology Co., Ltd. 24 animals were randomly divided into 4 groups: control group, low dose group, medium dose group, high dose group, 6 animals/group. All 24 animals were injected intraperitoneally with a combined intratracheal nebulization modeling reagent lipopolysaccharide (LPS). The specific steps are as follows:

Day 0: Lipopolysaccharide (0.8 mg/kg, 400 µL/kg) was given intraperitoneally by disposable microsyringe according to animal weight in each group.

Day 1 (16 h ±30 min after intraperitoneal injection): isoflurane inhalation anesthesia, each group of animals were given lipopolysaccharide (5 mg/kg, 1000 µL/kg) by intratracheal atomization. The main experimental steps are as follows: the animal is anesthetized by isoflurane inhalation, then fixed on a rat immobilizer placed at 45°, using a small animal anesthesia laryngoscope, pressing the root of the animal’s tongue, exposing the vocal cords, gently inserting a lung micro liquid nebulizer needle (blunt) extracting a quantitative amount of lipopolysaccharide (LPS) solution into the trachea, then quickly pushing the piston to nebulize the LPS solution into the lungs, quickly withdrawing the needle, removing the animal from the immobilizer with the head facing upward, and rotating it from side to side so that the LPS was distributed as evenly as possible throughout the lung lobes.

### 1.3 CEFFE Therapeutic Dose and Method

Day 1 animals were given intratracheal nebulization of the mold-making reagent (LPS) for about 1 h before CEFFE treatment or saline control was given according to the dose in Table 1 below.

TABLE 1

| Group number | Group | Administration volume (mL/kg) |
|---|---|---|
| 1 | Normal saline control group | 5 |
| 2 | Low dose group of CEFFE | 1 |
| 3 | Medium dose group of CEFFE | 2.5 |
| 4 | High dose group of CEFFE | 5 |

Route of administration: tail vein injection intravenous administration, using a disposable sterile syringe to draw the amount of drug for each animal, and the drug was administered by slow (about 10-60 seconds) injection in the tail vein. Dosing frequency and duration: Day1 animals were given the first dose after administering the mold-making reagent (LPS) by intratracheal nebulization for about 1 h; Day2 the second dose was administered; the interval between the two doses was about 24 h (±20 min).

### 1.4 Survival Record

The ratio of the number of animals surviving in each group to the total number of animals in each group during the test period was calculated, and the survival rate (%) = number of animals surviving in the group/total number of animals in the group × 100%.

### 1.5 Blood Gas Testing

Test time: the day after the end of the second administration (Day3: about 48 h after modeling).

Sample collection: rats were anesthetized by intraperitoneal injection of chloral hydrate (350 mg/kg, 100 mg/mL), the abdominal midline was incised longitudinally, the abdominal aorta was separated, and about 0.2 mL of arterial blood was collected using an arterial blood collector, and the blood collector was rubbed inside the palm of the hand for the syringe and inverted up and down for 5 seconds each, and the blood collector should never be pumped back, and the blood mixing process was performed.

Test method: after arterial blood collection, the needle was gently inserted into the blood injection port of the test card, the blood was pushed in slowly and filled the sample filling tube. When the blood reached the filling position, the adding was stopped, the cap was snapped, and the blood would automatically enter the test tube. the test card was inserted into the blood gas analyzer and waited for the test result.

Detection indexes: oxygen partial pressure PO2(mmHg), carbon dioxide partial pressure PCO2(mmHg), pH, blood oxygen saturation SO2%.

### 1.6 Assay for Bronchoalveolar Lavage Fluid (BALF)

Day 3: All animals were anesthetized by intraperitoneal injection of chloral hydrate (350 mg/kg, 100 mg/mL) and euthanized by abdominal aortic bleeding. Near-dead animals were euthanized by isoflurane anesthesia followed by abdominal aortic bleeding.

Sample collection: After the animal was euthanized, the skin and tissues of the neck and chest were cut open to expose the trachea, bronchi and lungs, and the right lung bronchus was separated and ligated. A suture was threaded under the trachea and a ½ incision was made between the tracheal cartilage rings at an appropriate location under the thyroid cartilage, and the tracheal cannula was slowly inserted along the incision into the airway to the left bronchus, and the cannula was tied and secured with the threaded suture at an appropriate location in the centripetal direction of the incision. The alveolar lavage was performed by slowly injecting 3 mL of PBS buffer with a syringe, and the lavage was repeated 3 times with a dwell time of about 10 s for each rinse, and the lavage fluid was collected in a centrifuge tube with suitable volume (not less than 2.1 mL).

BALF treatment: the collected lavage fluid was centrifuged at 4° C. for 20 min at approximately 2000 rpm. The supernatant was stored below -70° C. for later use, and the precipitate was resuspended in 1 mL of PBS buffer for white blood cell counting and classification.

Classification assay for white blood cell: The resuspended bronchoalveolar lavage fluid was used for white blood cell counting and classification using a fully automated hematocrit analyzer.

### 1.7 Histopathology

Animals in each group were dissected and tissues were preserved. During necropsy, animals were observed for abnormalities in the lungs, trachea and bronchi. The right lung was clipped and placed in 10% neutral buffered formalin solution for fixation, paraffin embedding, sectioning, filming, and HE staining for histomorphological observation of lung tissue using standard terminology for diagnosis, classification and pathology of lung tissue according to a 4-grading scale (slightly, mild, moderate, and severe).

### 1.8 Statistical Analysis

Data were statistically analyzed for significance of data differences by one-way ANOVA test using SPSS software and post hoc test using Bonferreni method, and results were expressed as mean ± standard deviation.

### 2. Results

### 2.1 CEFFE Treatment Effectively Improves Survival Rate of ARDS Rats

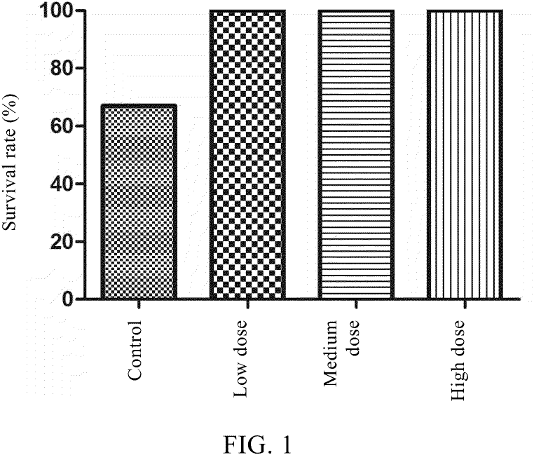

The survival of each group was recorded, and the survival rate of ARDS model rats is shown in FIG. 1. 2 rats died in the control group, and the survival rate was 67%, and no dead rats were seen in the CEFFE treatment group, indicating that CEFFE treatment effectively improved the survival rate of ARDS rats.

### 2.2 CEFFE Treatment Effectively Improves Respiratory Function and Hypoxemia in ARDS Rats

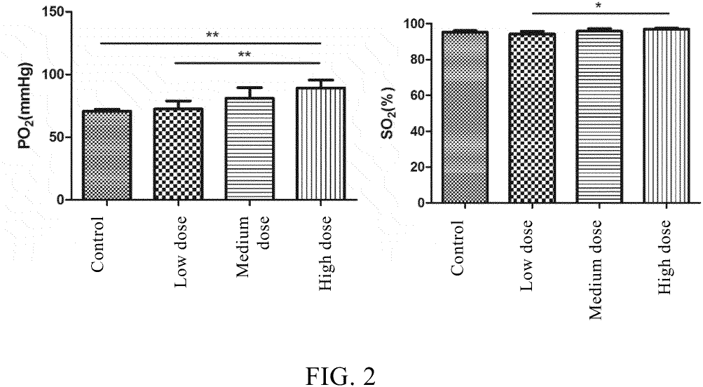

The results of arterial blood gas analysis are shown in Table 2 and FIG. 2.

TABLE 2

Arterial partial pressure of oxygen PO2 and blood oxygen saturation SO2% (M±SD) in model rats

| | PO₂ | SO₂ |
|---|---|---|
| Control | 70.75±1.5 | 95.25±0.96 |
| Low dose | 72.5±6.46 | 94.24±1.5 |
| Medium dose | 81±8.56 | 95.83±1.47 |
| High dose | 89.33±6.22 | 97±0.26 |

As seen in Table 2 and FIG. 2, PO2 was significantly impaired in the ADRS model rats. Oxygen partial pressure PO2 and oxygen saturation SO2% were significantly higher in the high-dose CEFFE treatment group compared with the control and low-dose treatment groups (P<0.05), indicating that CEFFE treatment effectively improved respiratory function and reduced hypoxemia in the model rats.

### 2.3 CEFFE Treatment Effectively Attenuated Inflammatory Cell Infiltration in Lung Tissues of ARDS Rats

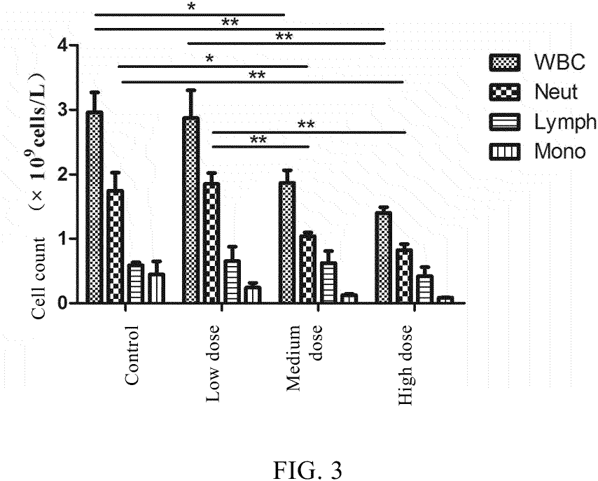

The inflammatory cell counts in BALF reflected the inflammation of lung tissue, and the inflammatory cell counts and classifications of bronchoalveolar lavage fluid were shown in Table 3 and FIG. 3.

TABLE 3

Inflammatory cell count and classification of bronchoalveolar lavage fluid (M±SD)

| | WBC | Neut | Lymph | Mono |
|---|---|---|---|---|
| Control | 2.96±0.617 | 1.74±0.58 | 0.59±0.09 | 0.45±0.41 |
| Low dose | 2.87±0.86 | 1.85±0.34 | 0.65±0.44 | 0.24±0.15 |
| Medium dose | 1.86±0.483 | 1.04±0.14 | 0.62±0.46 | 0.12±0.05 |
| High dose | 1.40±0.22 | 0.82±0.23 | 0.42±0.34 | 0.09±0.02 |

Note: WBC refers to white blood cells; Neut refers to neutrophil count; Lymph refers to lymphocytes; Mono refers to monocytes;

From Table 3 and FIG. 3, it has been seen that the number of white blood cells and neutrophils in the BALF of rats in the medium-dose and high-dose CEFFE treatment groups were significantly lower than those in the control group (P<0.05) in a dose-dependent manner, indicating that the infiltration of inflammatory cells in the lung was significantly reduced and that CEFFE treatment had the effect of reducing inflammation in the lungs of ARDS rats.

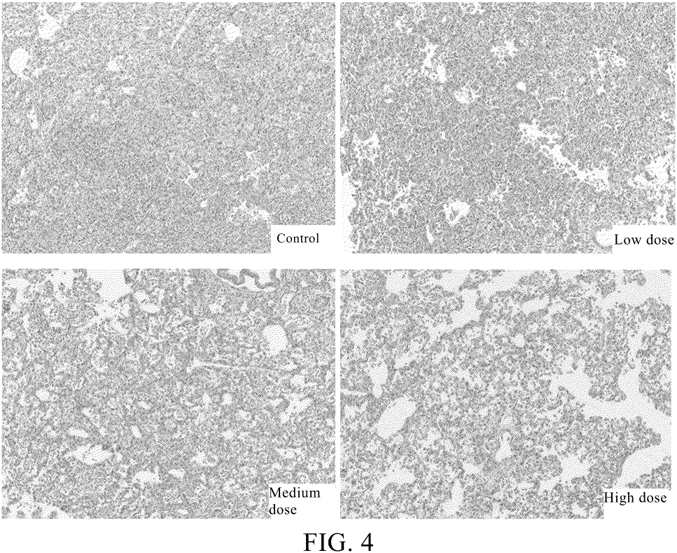

2.4 CEFFE treatment effectively improves pulmonary tissue injury in ARDS rats The results of histopathological HE staining are shown in FIG. 4, and the semi-quantitative evaluation of their pathological histology is shown in Table 4.

TABLE 4

Semi-quantitative evaluation of histopathology

| Group | Control | Low-dose | Medium- dose | High-dose |
|---|---|---|---|---|
| Number of animals examined | 4 | 4 | 6 | 6 |
| Inflammatory cell infiltration, interstitial/alveolar, multifocal | | | | |
| Mild | 0 | 0 | 2 | 4 |
| Moderate | 2 | 4 | 4 | 2 |
| severe | 2 | 0 | 0 | 0 |
| Alveolar macrophage aggregation, alveolar, multifocal | | | | |
| Mild | 1 | 1 | 1 | 4 |
| Moderate | 3 | 3 | 5 | 2 |
| Alveolar wall thickening, multifocal | | | | |
| Mild | 0 | 0 | 0 | 1 |
| Moderate | 4 | 4 | 6 | 5 |
| Fibrin-like material deposition, alveolar, multifocal | | | | |
| Slight | 1 | 2 | 3 | 4 |
| Mild | 2 | 2 | 3 | 0 |
| Bleeding, alveolar/perivascular, multifocal | | | | |
| Slight | 2 | 1 | 2 | 5 |
| Mild | 1 | 1 | 2 | 1 |
| Moderate | 1 | 0 | 0 | 0 |
| Alveolar dilatation, multifocal | | | | |
| Slight | 1 | 0 | 0 | 0 |
| Bronchial epithelial cell metaplasia, alveolar wall, multifocal | | | | |
| Slight | 1 | 0 | 0 | 0 |

As shown in Table 4 and FIG. 4, the control group showed neutrophil-based inflammatory cell infiltration, intra-alveolar macrophage aggregation, alveolar wall thickening, alveolar/perivascular hemorrhage, and intra-alveolar fibrin-like material deposition in the lung and bronchial interstitium/alveoli; compared with the model control group, CEFFE treatment reduced inflammatory cell infiltration and improved alveolar/perivascular hemorrhage in the medium-dose and high-dose groups, and there was a dose dependence, i.e., the improvement was more pronounced in the high-dose group than in the medium-dose group; in addition, the high-dose group also improved fibrin-like material deposition in the alveoli, and these results suggest that CEFFE treatment can effectively reduce pulmonary tissue injury in ARDS rats.

### Conclusion

Systemic inflammatory response syndrome (SIRS) is a clinical syndrome caused by infection or injury. Systemic inflammatory mediator release causing cytokine/inflammatory factor storm and inflammatory cell activation are its main contributing factors, causing apoptosis, shock, immune dysfunction, and organ damage. SIRS is triggered by infection and trauma, and the uncontrolled further development is multi-organ failure (MODS), and the lung is the most vulnerable organ during the evolution of this disease process; therefore, ALI/ARDS is an important disease symptom in SIRS patients.

The results of this experiment show that CEFFE treatment can effectively improve lung tissue damage caused by ALI/ARDS, reduce intrapulmonary inflammation, improve lung function, correct hypoxemia, and reduce mortality. The therapeutic effect of CEEFE on ALI/ARDS suggests its therapeutic value in improving SIRS and its potential therapeutic effect on other respiratory diseases.

All documents referred to in the present invention are incorporated by reference herein as if each document is individually incorporated by reference. Further, it should be understood that upon reading the above teaching of the present invention, various variations or modifications may be made to the present invention by those skilled in the art, and those equivalents also fall within the scope defined by the appended claims of the present application.

1. A method of (i) preventing and/or treating acute respiratory distress syndrome and/or acute lung injury, (ii) preventing and/or treating hypoxemia, (iii) improving pulmonary tissue inflammation, (iv) improving pulmonary tissue injury, (v) preventing and/or treating systemic inflammatory response syndrome, and (vi) preventing and/or treating multiple organ failure, comprising administering an effective amount of a composition to a subject in need thereof, wherein the composition comprises a cell-free fat extract.

2. The method of claim 1, wherein the preventing and/or treating of acute respiratory distress syndrome and/or acute lung injury comprises preventing and/or treating in one or more ways selected from the group consisting of :
(i) increasing blood oxygen level;
(ii) improving of lung tissue inflammation;
(iii) improving pulmonary tissue injury; and/or
(iv) improving respiratory function.

3. The method of claim 1, wherein the preventing and/or treating of hypoxemia comprises increasing blood oxygen level.

4. The method of claim 3, wherein the increasing the blood oxygen level comprises increasing the partial pressure of blood oxygen and/or increasing the saturation of blood oxygen.

5. The method of claim 1, wherein improving pulmonary tissue inflammation comprises reducing inflammatory cell infiltration in the lung.

6. The method of claim 1, wherein the improving pulmonary tissue injury comprises improving in one or more ways selected from the group consisting of:
(iv-1) reduction of intrapulmonary inflammatory cell infiltration;
(iv-2) inhibition of intra-alveolar macrophage aggregation;
(iv-3) inhibition of alveolar wall thickening;
(iv-4) inhibition of alveolar and/or perialveolar hemorrhage; and/or
(iv-5) inhibition of intra-alveolar fibrin-like material deposition.

7. The method of claim 1, wherein the cell-free fat extract is prepared by the following method:
(1) providing an fatty tissue raw material, cutting the fatty tissue raw material and rinsing it (e.g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano fat);
(5) centrifuging the emulsified fat mixture, thereby obtaining an intermediate liquid layer, i.e. a fat primary extract; and
(6) filtering and degerming the fat primary extract, thereby obtaining a cell-free fat extract.

8. The method of claim 5, wherein the inflammatory cells are selected from the group consisting of white blood cells, neutrophils, lymphocytes, monocytes, and combinations thereof.

9. The method of claim 1, wherein the composition or preparation is in the form of an oral preparation, an topical preparation or an injection preparation.

10. The method of claim 1, wherein the cell-free fat extract comprises one or more components selected from the group consisting of IGF-1, BDNF, GDNF, HGF, bFGF, VEGF, TGF-β1, HGF, PDGF, EGF, NT-3, GH, G-CSF, and combinations thereof.

11. The method of claim 10, wherein the cell-free fat extract comprises one or more features selected from the group consisting of:
in the cell-free fat extract, the concentration of the IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml;
in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml;
in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml;
in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml;
in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml;
in the cell-free fat extract, the concentration of TGF-β1 is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml;
in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml; and/or
in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

12. The method of claim 10, wherein the cell-free fat extract comprises one or more features selected from the group consisting of:
the weight ratio of the IGF-1 to VEGF is 20-100:1, preferably 30-70:1, more preferably 40-60:1, and most preferably 45-55:1;
the weight ratio of BDNF to VEGF is 2-20:1, preferably 4-15:1, more preferably 6-12:1, and most preferably 8-9.5:1;
the weight ratio of GDNF to VEGF is 2-20:1, preferably 4-15:1, more preferably 6-12:1, and most preferably 8.5-9.5:1;
the weight ratio of bFGF to VEGF is 0.2-8:1, preferably 0.5-5:1, more preferably 0.6-2:1, more preferably 0.8-1.6:1, and most preferably 1-1.5:1;
the weight ratio of TGF-β1 to VEGF is 1-20:1, preferably 1-15:1, more preferably 1-10:1, more preferably 2-8:1, more preferably 4-6:1;
the weight ratio of HGF to VEGF is 1-20:1, preferably 1-15:1, more preferably 1-10:1, more preferably 2-8:1, more preferably 4-5.5:1; and/or
the weight ratio of PDGF to VEGF is 0.1-3:1, preferably 0.2-2:1, more preferably 0.4-1.5:1, and most preferably 0.7-1.2:1.

13. A method for preparing cell-free fat extract, the method comprises the steps of:
(1) providing an fatty tissue raw material, cutting the fatty tissue raw material and rinsing it (e. g., with normal saline) to obtain a rinsed fatty tissue;
(2) centrifuging the rinsed fatty tissue to obtain a layered mixture;
(3) for the layered mixture, the upper oil layer and the lower water layer are removed, and collecting the intermediate layer (i. e. the fat layer containing fat cells);
(4) emulsifying the intermediate layer to obtain an emulsified fat mixture (also called nano fat);
(5) centrifuging the emulsified fat mixture, thereby obtaining an intermediate liquid layer, i.e. a fat primary extract; and
(6) filtering and degerming the fat primary extract, thereby obtaining a cell-free fat extract.

14. A cell-free fat extract, wherein the cell-free fat extract is prepared by the method according to claim 13.

15. (canceled)
